(19) United States
(12) Patent Application Publication
(10) Pub. No.: US 2022/0409522 A1
(43) Pub. Date: Dec. 29, 2022

(54) ORAL STRIPS AND METHODS OF MAKING SAME

(71) Applicant: Kinstate, Inc., Larkspur, CA (US)

(72) Inventor: JOSHUA KIRBY, Larkspur, CA (US)

(21) Appl. No.: 17/756,184

(22) PCT Filed: Nov. 19, 2020
(86) PCT No.: PCT/US2020/061380

§ 371 Date: May 18, 2022

Related U.S. Application Data
(60) Provisional application No. 62/937,909, filed on Nov. 20, 2019.

(51) Int. Cl. A61K 9/00; A61K 47/36; A61K 47/44

(52) U.S. Cl. CPC A61K 9/006; A61K 47/36; A61K 47/44

(57) ABSTRACT

The present invention generally relates to oral strips containing one or more active agents and methods of making oral strips containing an active agent. When the strip is placed in the mouth of a user, the active agent(s) is/are delivered to the user by way of oral mucosa.

### CLAIM OF PRIORITY

The present Application for Patent claims priority to Provisional Application No. 62/937,909 entitled “ORAL STRIPS AND METHODS OF MAKING SAME” filed Nov. 20, 2019 and assigned to the assignee hereof and hereby expressly incorporated by reference herein.

### BACKGROUND

### Field

The present invention generally relates to oral strips containing one or more active agents and methods of making oral strips containing an active agent. When the strip is placed in the mouth of a user, the active agent(s) is/are delivered to the user by way of oral mucosa.

### Background

Oral thin films made from pullulan, or other hygroscopic polysaccharides, can be used to deliver active ingredients to a user by way of the oral mucosa. It can be desirable to utilize active or non-active ingredients that are oils. This presents an issue as pullulan is oil-resistant resulting in the oily ingredients moving during and after the curing process. Over time, the agglomeration of the oil(s) can lead to lack of homogeneity in the film and leaching of the oil(s) out of the film altogether. Two methods are typically used to solve this issue: chemical modification of the pullulan—pullulan is modified to become accepting of oily ingredients; addition of synthetic surfactants to the film—synthetic surfactants provide a stable bridge between the pullulan and oil(s) and allows the oil(s) to remain in the film after the curing process. While effective, both of these methods result in a film that is, at least, partially synthetic and not all natural. It is desirable for the marketability of the product, the safety of the product, and for the health of the user, that thin films made from pullulan be entirely natural in their composition.

### SUMMARY

Some embodiments of the invention relate to an oral strip that can include water, one or more saponins, one or more oils with one or more active agents, and one or more polysaccharide(s).

Some embodiments of the invention relate to a device for delivering one or more active agents to a user that can include an oral strip, wherein the oral strip can include water, one or more saponins, one or more oils with one or more active ingredients, and one or more polysaccharide(s).

Some embodiments of the invention relate to a method for delivering one or more active agents to a user that can include placing an oral strip in the mouth of the user, wherein the oral strip can include water, one or more saponins, one or more oils with the one or more active agents, and one or more polysaccharide(s); wherein the active ingredient can be delivered through the oral mucosa of the user.

Some embodiments of the invention relate to a method of manufacturing an oral strip with one or more active agents that can include the steps of: creating a stable emulsion of one or more oils containing one or more active agents in water using one or more saponins; combining the stable emulsion with pullulan to create a slurry; and casting the slurry into a film wherein the oral strip can be formed.

Some embodiments of the invention relate to a method of manufacturing an oral strip with one or more active agents that can include the steps of: creating a stable slurry of one or more oils containing one or more active agents, water, one or more polysaccharides and one or more saponins; and casting the slurry into a film wherein the oral strip can be formed.

In some embodiments, the oral strip can be made of all-natural ingredients.

In some embodiments, the oral strip can be made of entirely all-natural ingredients.

In some embodiments, the oral strip can have a dissolution time of between 30 and 120 seconds.

In some embodiments, the oral strip can be capable of delivering one or more active agents in a period of time between 5 and 120 seconds,

In some embodiments, the oral strip can be capable of remaining free of microbiological activity for over 1 year.

In some embodiments, the oral strip can contain a dose of one or more active agents that is homogeneous throughout the oral strip allowing for the subdivision of the oral strip into smaller dosages.

In some embodiments, the strip contains beneficial volatile organic compounds that would otherwise evaporate under standard conditions.

### DETAILED DESCRIPTION

This disclosure provides compositions and methods related to oral strips containing one or more active agents. When the strip is placed in the mouth of a user, the one or more active agents are delivered to the user through the user's oral mucosa.

The oral strip can include, or can be manufactured with a solution including, water, saponin or a saponin-rich plant extract, one or more oils with one or more active agents, and/or one or more hygroscopic polysaccharide(s). The percent water can be 30-80%. In some embodiments, the percent water can be 52.5%-65.0%. For example, the percent water can be about 52.5, 55, 60, or 65%. The percent saponin or saponin-rich plant extract can be 0.2-10.0%. In some embodiments, the percent saponin or saponin rich plant extract can be 1.6%-3.0%. For example, the percent saponin or saponin-rich plant extract can be about 1.6, 2, 2.2, 2.4, 2.6, 2.8, or 3%. The percent oil can be 1-40%. In some embodiments, the percent oil can be 10.0%-15.0%. For example, the percent oil can be about 10, 11, 12, 13, 14, or 15%. The pullulan can be pullulan that is natural in origin and unmodified. The percent hygroscopic polysaccharide can be 1-40%. In some embodiments, the percent pullulan can be 5.0%-17.0%. For example, the percent pullulan can be about 5, 6, 7, 8, 9, 10, 11, 12, 13, 14, 15, 16 or 17%. The oral strip can be made of all-natural ingredients.

In some embodiments, the oil can be a vegetable or botanical oil. Examples of oils that can be used include, but are not limited to, hemp oil, cannabis oil, MCT oil, coconut oil, avocado oil, peppermint oil, mango oil, sunflower oil, cottonseed oil, citrus oil, safflower oil, rice bran oil, cocoa butter, palm fruit oil, palm kernel oil, soybean oil, rapeseed oil, corn oil, sesame oil, walnut oil, flaxseed oil, jojoba oil, castor oil, grape seed oil, peanut oil, olive oil, algae oil, mushroom oil, or the like, or any combination thereof.

Saponins are a subclass of terpenoids and can also be referred to a triterpene glycosides. Saponins have been reported in more than 100 families of plants and in a few marine sources such as star fish and sea cucumber. Examples of saponin-rich plant extract sources used in the invention can include, but are not limited to soap bark tree (Quillaja saponaria), Mojave yucca (Yucca schidigera), licorice (Glycyrrhiza species), ginseng (Panax species), fenugreek (Trigonellafoenum graceum), alfalfa (Medicago sativa), horse chestnut (Aesculus hippocastanum), soapwort (Saponaria officinaux), gypsophila genus (Gypsophila paniculata) and sarsaparilla (Smilax species) or other sources, and/or the like, and/or combinations of the same. Saponins of use can include, but are not limited to, soyasaponin A(1), soyasaponin A(2), soyasaponin I, soyasaponin B, deacetylated soyasaponin, acetylated soyasaponin, soyasaponin II, soyasaponin III and soyasapogenol B monoglucuronide. In preferred embodiments, the saponins of use may be present in an extract, homogenate, finely ground powder, or other derivative of plant matter, such as beans, peas, nuts or other plant parts. In some embodiments, the relative proportions of plant extracts, homogenates, finely ground powders, or derivatives can be selected to optimize the content of one or more saponins of use in the composition for oral delivery of an active agent.

The one or more active agents can be any essential plant extract that has a concentration of active agent between 25% and 99.9% of the total mass. The active agents can be therapeutically beneficial molecules or combinations of the same such as delta-9-tetrahydrocannabinol, delta-8-tetrahydrocannabinol, cannabinol, cannabidiol, tetrahydrocannabinolic-acid, cannabidiolic-acid, cannabigerol, cannabigerolic-acid, cannabichromene, tetrahydrocannabivarin, cannabivarin and/or any other cannabinoid, terpene, or naturally occurring plant compound.

In some embodiments, the composition can include one or more terpenes or a terpene blend. Exemplary terpenes include, but are not limited to: 3-carene; alpha pinene; beta caryophyllene; beta pinene; bisabolol; borneol; camphene; camphor; caryophyllene; caryophyllene oxide; cedrene; cedrol; citronellol; delta 3 carene; eucalyptol; fenchol; fenchone; geraniol; geranyl acetate; guaiol; humulene; isoborneol; isopulegol; limonene; linalool; menthol; myrcene; nerol; nerolidol; ocimene; phellandrene; phytol; pulegone; sabinene; terpinene; terpineol; terpinolene; and valencene.

The polysaccharide used in the invention can be from a natural substance. Examples of such polysaccharides include, but are not limited to, polymers obtained from natural substances such as acacia gum, gum arabic, sodium alginate, casein, xanthan gum, guar gum, dextran, tragacanth gum, starch, pullulan, pectin, and/or the like.

In some embodiments, the saponin can act as an emulsifier. In such embodiments, the hydrophilic-lipophilic balance (HLB) of the saponin can be between about 8-17. For example, the HLB can be about 13.5.

In some embodiments, the strip can contain beneficial volatile organic compounds that evaporate under standard conditions if not contained in the strip. Examples of such ingredients can include, but not be limited to, the terpenes listed above as well as any other VOC capable of conferring a benefit on the user.

In some embodiments, the strip is made of entirely all-natural ingredients. As used herein, the term “all-natural” means that the ingredient does not include any synthetic or artificial components.

In some embodiments, the strip made of entirely all-natural ingredients has similar or improved physical characteristics compared to a strip made of one or more synthetic ingredients. The physical characteristic can include tensile strength, percent elongation, elastic modulus, folding endurance, thickness, disintegration time, uniformity of drug content, potency, moisture content, weight, pharmacokinetics, etc.

References that provide characteristics of systems made of synthetic ingredients include: Nayak BS, International Journal of Pharmaceutics & Drug Analysis; Vol. 5, Issue 10, 2017; 399-405; Mannila J, Järvinen T, Järvinen K, Tervonen J, Jarho P. “Sublingual administration of Delta9-tetrahydrocannabinol/beta-cyclodextrin complex increases the bioavailability of Delta9-tetrahydrocannabinol in rabbits.” Life Sci. 2006; 78(17):1911-1914; Narang N, Sharma J. “Sublingual Mucosa as a Route for Systemic Drug Delivery.” Int J of Pharm Sci, Vol 3, Suppl 2, 2011, 18-22; and Katz M, Barr M. “A study of sublingual absorption. I. Several factors influencing the rate of adsorption.” J Am Pharm Assoc Am Pharm Assoc. 1955; 44(7):419-423. Each of the foregoing is hereby incorporated by reference in its entirety.

Some embodiments of the invention relate to methods of manufacturing an oral strip with one or more active agents. The method can include the steps of creating a stable emulsion of one or more oils containing one or more active agents in water using one or more saponins; combining the stable emulsion with a polysaccharide to create a slurry; and casting the slurry into an oral strip.

The step of creating a stable emulsion of one or more oils containing an active agent can include the use of a high-speed mixing apparatus such as an immersion blender, homogenizing wand, emulsifying mixer, a mixing apparatus that utilizes sound energy such as a sonicating wand, a mixing apparatus that utilizes high pressure energy such as a microfluidizer, and/or the like. The mixture can be prepared at room temperature. The emulsion can be mixed for a period of 2 minutes to 24 hours depending on application. In some embodiments, the method can include multiple successive mixing sessions to achieve a stable emulsion.

The step of combining the stable emulsion with pullulan to create a slurry can include maintaining both the emulsion and the polysaccharide(s) at room temperature and combining at a speed of between 30-120 RPM using a planetary mixer for 2 min-24 hrs. Alternatively, the step can include combining at a speed of between 1000-5000 RPM using an emulsifying mixer.

The step of combining the stable emulsion with pullulan to create a slurry can further include adding one or more additional ingredients. The one or more additional ingredients can include natural flavorings such as fruit and herbal extracts and/or sweeteners; natural colorings such as fruit, vegetable and/or herbal powders and/or extracts; humectant(s) such as glycerin, lecithin, beeswax, honey, aloe vera and/or other natural humectants; natural phospholipids such as sunflower lecithin, soy lecithin, egg lecithin, bovine lecithin, and/or other natural phospholipids; and natural gums including acacia gum, xanthan gum, agar, gellen gum, guar gum, locust bean gum, and/or other natural gums.

The step of casting the slurry into an oral strip can include mold casting; die casting; roll to roll casting equipment that utilizes doctor-blades, coating bars, or slot-dies; automatic and/or manual drawdown coating equipment; doctor-blade casting equipment; sputter coating equipment; electrospinning equipment; spin coating, dip coating, bar coating, spray deposition and or any method practicable for creating a film of uniform thickness on a substrate.

The oral strip can be any size or shape that would reasonably fit under a user's tongue such as a quadrilateral that is between 0.125″×0.125″-1.5″×1.5″, non-ridged shapes (such as crescent moons, horseshoes) or round shapes (such as ovals or circles). The strips may be of a thickness between 0.0001″ and 0.5″. In some embodiments, the strips can have a thickness of 0.0002, 0.0005, 0.001, 0.005, 0.01, 0.05, 0.1, or 0.25″.

In some embodiments of the invention, as the slurry is cast into strips and cured, the natural saponins stabilize the position of the oil(s) so that the homogeneity of the ingredients achieved during the mixing stage remains high and the oil(s) ultimately remain in the film.

The oral strip can have a final active ingredient concentration of about between 0.01% and 35%. In some embodiments, the final active ingredient concentration can be 0.02, 0.05, 0.1, 0.5, 1, 3, 5, 10, 15, 20, 25, or 30%.

Some embodiments of the invention relate to the use of the strips to treat a medical condition. The medical condition can include insomnia, anxiety, intractable pain, Crohn's disease, Alzheimer's disease, appetite loss, cancer, eating disorders, epilepsy, glaucoma, schizophrenia, PTSD, multiple sclerosis, muscle spasms, nausea, wasting syndrome, and/or the like.

The strips of the invention can include characteristics such as:
Varying natural colors (orange, pink, red, salmon, blue, green, teal, purple, etc).
Varying natural flavors (mint, spearmint, cinnamon, citrus, mango, menthol, chamomile, turmeric, tarragon, watermelon, basil, etc).
An absorption time of between 10 seconds and 1,800 seconds or between 10 seconds and 600 seconds. In some embodiments, the absorption time can be 20, 30, 40, 50, 60, 90, 120, 150, 180, 210, 240, 270, 300, 350, 400, 450, 500, or 550 seconds.
A degradation time of between 1 second and 600 seconds or between 1 second and 300 seconds. In some embodiments, the degradation time can be 2, 3, 5, 10, 15, 20, 30, 60, 90, 120, 150, 180, 210, 240, or 270 seconds.
A potency of between 0.1 mg and 30 mg or between 0.1 mg and 20 mg. In some embodiments the potency can be 0.2, 0.5, 1, 2, 3, 4, 5, 10, 15, 20 or 25 mg.
A moisture content between 0%-18% or 0%-10% in its cured form. In some embodiments the moisture continent can be 0.1, 0.25, 0.5, 1, 2, 3, 4, 5, 6, 7, 8 or 9%.
A weight of between 5 mg and 173 mg in its cured form or 5 mg-75 mg. In some embodiments the weight in its cured form can be 10, 15, 20, 25, 30, 35, 40, 45, 50, 55, 60, 65 or 70 mg.
A dissolution time of between 1 and 120 seconds or between 1 and 60 seconds. In some embodiments the dissolution time can be 2, 3, 5, 10, 15, 20, 30, 40, or 50 seconds.
Delivery time of active agent(s) between 5 and 120 seconds or 5 seconds to 90 seconds. In some embodiments, the delivery time of active agent(s) can be 6, 7, 8, 9, 10, 15, 20, 30, 40, 50, 60, 70, or 80 seconds.
Capacity to remain free of microbiological activity for over 1 year.
Homogeneity of dose of active agent(s) throughout the oral strip allowing for the breaking apart of the oral strip for smaller dosages.

### EXAMPLES

The present invention will be more specifically described by means of the following Examples which, however, are not intended to limit the scope of the present invention.

### Example 1

A film made from natural ingredients as described herein was compared to a film made with traditional ingredients. A thin film formula that utilized a blend of 2:1 Tween 20 to Span 20 with a combined (hydrophilic-lipophilic balance) HLB of 14 and substituted those ingredients 1:1 with Quillaja Extract at 13.5 HLB without making any other changes to the formulation or process. The end result was no noticeable change in the formation, characteristics, functionality, or stability of the emulsion or resulting film.

### Example 2

A film made with natural ingredients as described herein is compared to a film made with traditional ingredients. Tensile strength is measured and compared quantitatively and with respect to user experience/perception. Tests show high similarity with respect to this factor.

### Example 3

A film made with natural ingredients as described herein is compared to a film made with traditional ingredients. Percent elongation is measured and compared quantitatively and with respect to user experience/perception. Tests show high similarity with respect to this factor.

### Example 4

A film made with natural ingredients as described herein is compared to a film made with traditional ingredients. Elastic modulus is measured and compared quantitatively and with respect to user experience/perception. Tests show high similarity with respect to this factor.

### Example 5

A film made with natural ingredients as described herein is compared to a film made with traditional ingredients. Folding endurance is measured and compared quantitatively and with respect to user experience/perception. Tests show high similarity with respect to this factor.

### Example 6

A film made with natural ingredients as described herein is compared to a film made with traditional ingredients. Thickness is measured and compared quantitatively and with respect to user experience/perception. Tests show high similarity with respect to this factor.

### Example 7

A film made with natural ingredients as described herein is compared to a film made with traditional ingredients. Disintegration time is measured and compared quantitatively and with respect to user experience/perception. Tests show high similarity with respect to this factor.

### Example 8

A film made with natural ingredients as described herein is compared to a film made with traditional ingredients. Uniformity of drug content is measured and compared quantitatively and with respect to user experience/perception. Tests show high similarity with respect to this factor.

### Example 9

A film made with natural ingredients as described herein is compared to a film made with traditional ingredients. Potency is measured and compared quantitatively and with respect to user experience/perception. Tests show high similarity with respect to this factor.

### Example 10

A film made with natural ingredients as described herein is compared to a film made with traditional ingredients. Moisture content is measured and compared quantitatively and with respect to user experience/perception. Tests show high similarity with respect to this factor.

### Example 11

A film made with natural ingredients as described herein is compared to a film made with traditional ingredients. Weight is measured and compared quantitatively and with respect to user experience/perception. Tests show high similarity with respect to this factor.

### Example 12

A film made with natural ingredients as described herein is compared to a film made with traditional ingredients. Pharmacokinetics are measured and compared quantitatively and with respect to user experience/perception. Tests show high similarity with respect to this factor.

### Example 13

The following formula describes the typical ranges of the mentioned ingredients for the described invention to function:

Water-52.5%-65.0%

Saponin-rich plant extract—1.6%-3.0%

Oil(s)-10.0%-15.0%

Natural Pullulan-10.0%-17.0%

### Example 14

The following formula describes the typical ranges of the mentioned ingredients for the described invention to function:

Water-52.5%-65.0%

Saponin-rich plant extract-1.6%-3.0%

Cannabinoid oils-10.0%-15.0%

Terpene Blend—0.5%-5.0%

Natural Pullulan—5.0%-17.0%

Natural Gum Arabic—7.5%-16.0%

Natural Tapioca Starch—2.5%-12.5%

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. An oral strip comprising water, one or more saponins, one or more oils with one or more active agents, and one or more polysaccharide(s).

2. (canceled)

3. A method for delivering one or more active agents to a user comprising
placing an oral strip in the mouth of the user,
wherein the oral strip comprises water, one or more saponins, one or more oils with the one or more active agents, and one or more polysaccharide(s); wherein the active ingredient is delivered through the oral mucosa of the user.

4. A method of manufacturing an oral strip with one or more active agents comprising the steps of:
creating a stable slurry of one or more oils containing one or more active agents, water, one or more polysaccharides and one or more saponins;
casting the slurry into a film wherein the oral strip is formed.

5. The oral strip of claim 1 consisting of all all-natural ingredients.

6. The oral strip of claim 1, wherein the oral strip has a dissolution time of between 30 and 120 seconds.

7. The oral strip of claim 1, wherein the oral strip is capable of delivering one or more active agents in a period of time between 5 and 120 seconds,

8. The oral strip of claim 1, wherein the strip is capable of remaining free of microbiological activity for over 1 year.

9. The oral strip of claim 1, wherein the strip contains a dose of the one or more active agents that is homogeneous throughout the oral strip allowing for the breaking apart of the oral strip for smaller dosages.

10. The oral strip of claim 1, wherein the strip contains beneficial volatile organic compounds that evaporate under standard conditions if not contained in the strip.

11. The method of claim 3, wherein the oral strip has a dissolution time of between 30 and 120 seconds.

12. The method of claim 3, wherein the oral strip is capable of delivering one or more active agents in a period of time between 5 and 120 seconds.

13. The method of claim 4, wherein the strip is capable of remaining free of microbiological activity for over 1 year.

14. The method of claim 4, wherein the strip contains a dose of the one or more active agents that is homogeneous throughout the oral strip allowing for the breaking apart of the oral strip for smaller dosages.

15. The method of claim 4, wherein the strip contains beneficial volatile organic compounds that evaporate under standard conditions if not contained in the strip.
